(19) United States
(12) Patent Application Publication
(10) Pub. No.: US 2008/0113367 A1
(43) Pub. Date: May 15, 2008

(54) CLIP BASED SAMPLING OF SEED FOR THE REMOVAL OF SPECIFIC SEED TISSUE OR STRUCTURES FOR SEED ANALYSIS

(75) Inventors: STEVEN M. BECKER, JOHNSTON, IA (US); JASON COPE, ANKENY, IA (US)

Correspondence Address: MCKEE, VOORHEES & SEASE, P.L.C.;ATTN: PIONEER HI-BRED, 801 GRAND AVENUE, SUITE 3200, DES MOINES, IA, 50309-2721, US

(73) Assignee: PIONEER HI-BRED INTERNATIONAL, INC., JOHNSTON, IA (US)

(21) Appl. No.: 11/939,381

(22) Filed: Nov. 13, 2007

Related U.S. Application Data
(60) Provisional application No. 60/865,563, filed on Nov. 13, 2006.
(60) Provisional application No. 60/865,554, filed on Nov. 13, 2006.

(51) Int. Cl. B26B 17/00; A01G 3/00; B26B 29/06; C12Q 1/00; C12Q 1/68

(52) U.S. Cl. 435/6; 30/124; 30/286; 435/4

(57) ABSTRACT

Apparatus, methods, and systems for useful sampling of seed, wherein viability is maintained, are disclosed. Seed from one generation in a plant advancement experiment is individually sampled by removal and collection of tissue from the seed using a hand-held and manually operated tool having one or more cutting edges. The tissue is then processed to derive one or more biochemical, genetic, or phenotypic characteristic of the seed before a decision is made whether to utilize that seed further in a plant advancement experiment or other plant research and development. In some embodiments of the method, the sampling is controlled to remove a useful amount of tissue for analytical purposes without significant effect on viability potential of the sampled seed. In some embodiments, the sampling is controlled to deter contamination of the sample. In some embodiments, the seed is held in pre-determined orientation to facilitate efficient and accurate sampling.

### CROSS-REFERENCE TO RELATED APPLICATIONS

This application claims priority under 35 U.S.C. § 119 of provisional applications U.S. Ser. No. 60/865,554 and U.S. Ser. No. 60/865,563, both filed Nov. 13, 2006, which applications are hereby incorporated by reference in their entireties.

### I. BACKGROUND OF THE INVENTION

A. Field of the Invention

The present invention relates to obtaining tissue samples from individual seed in an efficient way.

B. Problems in the Art

It is conventional practice in plant breeding or plant advancement experiments to grow plants from seed of known parentage. The seed are planted in experimental plots, growth chambers, greenhouses, or other growing conditions in which they are either cross pollinated with other plants of known parentage or self pollinated. The resulting seed are the offspring of the two parent plants or the self pollinated plant, and are harvested, processed and planted to continue the plant breeding cycle. Specific laboratory or field-based tests may be performed on the plants, plant tissues, seed or seed tissues, in order to aid in the breeding or advancement selection process.

Generations of plants based on known crosses or self pollinations are planted and then tested to see if these lines or varieties are moving towards characteristics that are desirable in the marketplace. Examples of desirable traits include, but are not limited to, increased yield, increased homozygosity, improved or newly conferred resistance and/or tolerance to specific herbicides and/or pests and pathogens, increased oil content, altered starch content, nutraceutical composition, drought tolerance, and specific morphological based trait enhancements.

As can be appreciated and as is well known in the art, these experiments can be massive in scale. They involve a huge labor force ranging from scientists to field staff to design, plant, maintain, and conduct the experiments, which can involve thousands or tens of thousands of individual plants. They also require substantial land resources. Plots or greenhouses can take up thousands of acres of land. Not only does this tie up large amounts of land for months while the plants germinate, grow, and produce seed, during which time they may be sampled for laboratory or field testing, but then the massive amounts of seed must be individually tagged, harvested and processed.

A further complication is that much of the experimentation goes for naught. It has been reported in the literature that some seed companies discard 80-90% of the plants in any generation early on in the experiment. Thus, much of the land, labor and material resources expended for growing, harvesting, and post-harvest processing ultimately are wasted for a large percentage of the seed.

Timing pressures are also a factor. Significant advances in plant breeding have put more pressure on seed companies to more quickly advance lines or varieties of plants for more and better traits and characteristics. The plant breeders and associated workers are thus under increasing pressure to more efficiently and effectively process these generations and to make more and earlier selections of plants which should be continued into the next generation of breeding.

Therefore, a movement towards earlier identification of traits of interest through laboratory based seed testing has emerged. Seed is non-destructively tested to derive genetic, biochemical or phenotypic information. If traits of interest are identified, the selected seed from specific plants are used either for further experiments and advancement, or to produce commercial quantities. Testing seed prevents the need to grow the seed into immature plants, which are then tested. This saves time, space, and effort. If effective, early identification of desirable traits in seed can lead to greatly reducing the amount of land needed for experimental testing, the amount of seed that must be tested, and the amount of time needed to derive the information needed to advance the experiments. For example, instead of thousands of acres of plantings and the subsequent handling and processing of all those plants, a fraction of acres and plants might be enough. However, because timing is still important, this is still a substantial task because even such a reduction involves processing, for example, thousands of seed per day.

A conventional method of attempting non-lethal seed sampling is as follows. A single seed of interest is held with pliers above a sheet of paper laid out on a surface. A small drill bit is used to drill into a small location on the seed. Debris removed by the drill bit from the seed is collected on the sheet of paper. The paper is lifted and the debris is transferred to a test tube or other container. It is thus collected and ready for laboratory analysis. This method is intended to be non-lethal to the seed. However, the process is slow. Its success and effectiveness depends heavily on the attention and accuracy of the worker. Each single seed must be manually picked up and held by the pliers. The drilling is also manual. Care must be taken with the drilling and the handling of the debris. Single containers, e.g. the individual test tubes, must then be handled and marked or otherwise tracked and identified. Additionally, the pliers and drill must be cleaned between the sampling of each seed. There can be substantial risk of contamination by carry-over from sample to sample and the manual handling. Also, many times it is desirable to obtain seed material from a certain physiological tissue of the seed. For example, with corn seed, it may be desirable to take the sample from the endosperm. In such cases, it is not trivial, but rather is time-consuming and somewhat difficult, to manually grasp a small corn seed in such a way to allow the endosperm to be oriented to expose it for drilling. Sampling from other seed structures such as the seed germ must be avoided because sampling from such regions of the seed negatively impacts germination rates. Sometimes it is difficult to obtain a useful amount of sample with this method. In summary, sampling from seed relies heavily on the skill of the worker and is relative to throughput and accuracy, including whether the procedure gives the seed a good chance at germination. These issues are amplified when a worker is charged with processing many seed a day.

Another example of non-lethally obtaining tissue samples from corn seed for laboratory analysis is disclosed at V. Sangtong, E. C. Mottel, M. J. Long, M. Lee, and M. P. Scott, Serial Extraction of Endosperm Drillings (SEED)—A Method for Detecting Transgenes and Proteins in Single Viable Maize Kernels, Plant Molecular Biology Reporter 19: 151-158, June 2001, which is incorporated by reference herein. It describes use of a hand-held rotary grinder to grind off particles, called “drillings,” from the kernel and collection of the particles to test for the presence of certain genes. Particles are directed to individual wells of a multi-well tray or plate for identification and preparation for subsequent testing and analysis. However, this method also requires manual grasping and orientation of each individual seed relative to the grinder. It too, is time consuming and somewhat cumbersome. It also relies on the skill of the worker. This method raises issues of throughput, accuracy, whether a useful amount of sample is obtained, and contamination. The grinder must be thoroughly cleaned between each sample in order to prevent contamination.

As evidenced by these examples, present conventional seed analysis methods, such as is used in genetic, biochemical, or phenotypic analysis, require at least a part of the seed to be removed and processed. In removing some seed tissue, various objectives may need to be met. These may include one or more of the following objectives:

(a) maintain seed viability post-sampling;

(b) obtain at least a minimum required sample amount, without affecting viability;

(c) obtain a useful amount of sample from a specific location on the seed, often requiring the ability to orient the seed in a specific position for sampling;

(d) maintain a particular throughput level for efficiency purposes;

(e) reduce or virtually eliminate contamination between samples; and

(f) allow for the tracking of separate samples and their correlation to other samples in a group.

(a) Viability

With regard to maintaining seed viability, it may be critical in some circumstances that the seed sampling method and apparatus not damage the seed in such a way that seed viability is reduced or lost entirely. It is often desirable that such analysis be non-lethal to the seed, or at least result in a substantial probability that the sampled seed will germinate (e.g. no significant decrease in germination potential) so that it can be grown into a mature plant. For some analysis, seed viability does not need to be maintained, in which case larger samples can often be taken. The need for seed viability will depend on the intended use of the seeds post-sampling.

(b) Sample Amount

It is desirable to obtain a useful amount of sample. To be useful, in some applications it must be above a certain minimum amount necessary in order to perform a given test and obtain a meaningful result. Different tests or assays require different sample amounts. It may be equally important to avoid taking too much tissue for a sample, because a sample that is too large may reduce germination potential of a seed, which may be undesirable. Therefore, it is desirable that sampling apparatus and methods allow for variation in the amount of sample taken from any given seed.

(c) Sample Location

A useful sample amount also can involve sample location accuracy. For example, in some applications the sample must come only from a certain location or from certain tissue. Further, it is difficult to handle small particles like many seed. It is also difficult to accurately position and orient seed. On a corn seed, for example, it may be important to sample the endosperm tissue, and orient the corn seed for sampling that particular tissue. Therefore, it is desirable that sampling apparatus and methods are adapted to allow for location-specific sampling, which may include specific seed orientation methods.

(d) Throughput

A sampling apparatus and methodology must consider the throughput level that supports the required number of samples being taken in a time efficient manner. For example, some situations involve the potential need to sample thousands, hundreds of thousands, or even millions of seed per year. Taking the hypothetical example of a million seed per year, and a 5-day work week, this would average nearly four thousand samples per day for each working day of a year. It is difficult to meet such demand with lower throughput sampling methods. Accordingly, higher throughput, automatic or even semi-automatic methods may be desirable.

(e) Avoiding Contamination

It is desirable that a sampling methodology and apparatus not be prone to cross-contamination in order to maintain sample purities for subsequent analytical testing procedures. This can involve not only sample location accuracy, such that a sample from a given location is not contaminated with tissue from a different location, but also the method of sampling and handling of each individual sample, ensuring no contamination between samples.

(f) Tracking Samples

Efficient processing of seed and samples removed from seed presents a variety of issues and challenges, especially when it is important to keep track of each seed, each sample, and their correlation to each other, or to other samples within a batch. Accordingly, it is desirable that a sampling apparatus and methodology allow for easy tracking of seed and samples.

Conventional seed sampling technologies do not address these requirements sufficiently, resulting in pressures on capital and labor resources, and thus illustrate the need for an improvement in the state of the art. The current methods are relatively low throughput, have substantial risk of cross-contamination, and tend to be inconsistent because of a reliance on significant manual handling, orienting, and removal of the sample from the seed. This can affect the type of sample taken from the seed and the likelihood that the seed will germinate. There is a need to eliminate the resources current methods require for cleaning between samples. There is a need to reduce or minimize cross-contamination between samples by carry-over or other reasons, or any contamination from any source of any sample. There is also a need for more reliability and accuracy. Accordingly, there is a need for methodologies and their corresponding apparatus which provide for seed sampling that accomplishes one or more of the following objectives:

(a) maintains seed viability post-sampling;

(b) obtains at least a minimum required sample amount, without affecting viability;

(c) obtains a sample from a specific location on the seed;

(d) maintains a particular throughput level for efficiency purposes;

(e) reduces or virtually eliminate contamination between samples; and

(f) allows for the tracking of separate samples and their correlation to other samples in a group.

Some of these objectives that are desirable when sampling seed can be conflicting and even antagonistic. For example, obtaining a useful sample amount while maintaining seed viability requires taking some seed tissue, but not too much tissue. High throughput methodologies may require relatively rapid operation but with relatively high accuracy and low contamination risk, such that they must be done more slowly than is technically possible. These multiple objectives have therefore existed in the art and have not been satisfactorily addressed or balanced by the currently available methods and apparatuses.

As can be appreciated, semi- or full automation of processes can often meet at least some of the above-listed objectives (a)-(f). However, automation also involves substantial cost, complexity, and machines, which are not portable and occupy substantial space. Substantial resources are also consumed by training, support, and maintenance. These types of additional factors can therefore also be a concern. It is not a trivial matter to balance multiple factors and issues relative to the objectives when considering improved seed sampling methods.

For example, in situations where relatively large numbers of seed are to be sampled in a limited amount of time, automated solutions are many times sought out or investigated. However, automation introduces not only cost and complexity, but also practicality issues. There are reasons, therefore, to develop new seed sampling methods that satisfy as many of the seed sampling objectives listed above as possible.

### II. SUMMARY OF THE INVENTION

It is therefore an embodiment of the present invention to provide an apparatus, method, or system which improves over the problems and deficiencies in the state of the art.

Further embodiments of the present invention include an apparatus, method, or system which:
a. promotes acceptable through-put of collecting samples from plural individual seed;
b. promotes sampling with relatively high germination rates for the sampled seed;
c. promotes relatively consistent, accurately measured samples of useful amounts;
d. provides for efficient biochemical, genetic, or phenotypic evaluation of samples;
e. promotes more efficient selection during a plant advancement experiment;
f. avoids contamination of samples and cross-contamination between samples;
g. promotes sampling which is not detrimental to biochemical, genetic, or phenotypic testing;
h. promotes accuracy, consistency and reliability of sampling;
i. is flexible in application to a variety of seed types and a variety of sampling tasks;
j. is efficient and robust;
k. can reduce the amount of field space and the number of samples that are needed for analysis and for shipping in plant advancement experiments;
l. reduces materials required for management of plant nurseries;
m. reduces overall labor resource requirements;
n. is not complex;
o. is economical;
p. is portable;
q. is not burdensome or difficult regarding training and application;
r. reduces plant tracking and tagging efforts; and
s. improves ergonomics of the sampling process.

One embodiment of the present invention is a method whereby seed from one generation in a plant advancement experiment is individually sampled by removal and collection of a useful amount of tissue from the seed by use of clipping or similar action by one or more blades without significant reduction in germination potential or viability of the sampled seed. The tissue is then processed to derive one or more biochemical, genetic, or phenotypic characteristic of the seed before a decision is made whether to utilize that seed further in the plant advancement experiment or other plant-based research. This allows a decision to be made as soon as the seed matures in a given generation, as opposed to harvesting the seed, planting it in an experimental plot, and then testing the immature plant by scientific analyses such as genetic testing for the presence of traits that influence seed components such as oils, proteins, starches, or other such procedures. This allows for earlier determination of which offspring will be selected to continue in the experiment. It further allows the benefit of reducing the amount of land required for experimental plots. In a single nursery it essentially reduces the amount of land required to screen thousands of plants by identifying the 10%-20% of the seed that are needed for advancement. As a result, no field sampling is required because decisions are not based on plant tissue. The seed sampling technique allows for a quick and efficient decision on which seed carry desirable genetic traits or other characteristics.

Another embodiment of the invention is a method, apparatus, and system for effectively obtaining samples of useful amounts from seed while maintaining a high germination potential for the seed.

Another embodiment of the invention deters or minimizes contamination in sample taking and handling.

These and other embodiments of the present invention will become apparent with reference to the accompanying specification and claims.

### III. BRIEF DESCRIPTION OF THE DRAWINGS

Appended to this description are several drawings and illustrations that will be referred to herein and which are incorporated by reference into this description.

FIG. 1 illustrates a single seed clipping device and method according to an embodiment of the present invention.

FIGS. 2A and B illustrate a single seed positioning and clipping tool, and a sample indexing system according to another embodiment of the present invention.

### IV. DETAILED DESCRIPTION OF EXEMPLARY EMBODIMENTS

### A. Overview

For a better understanding of the invention, examples of how aspects of the invention can be practiced will now be described in detail. It is to be understood that these are but several forms the invention can take and do not limit the invention.

Frequent reference will be taken to the accompanying drawings. Reference numerals and/or letters will be used to indicate certain parts and locations throughout the drawings. The same reference numerals will be used to indicate the same parts or locations unless otherwise indicated.

The context of these specific examples will be with respect to kernels of corn. It is to be understood, however, that this example is only intended to illustrate one application of the invention. The invention can be utilized for other seed and other objects. The range of sizes can vary as well as the nature of the object. As will be understood by one of skill in the art, the embodiments of the invention will be used with seed that are of convenient size to be sampled. Some seed are extremely fine and small, somewhat like dust particles or grains of salt, while others are particularly large and hard, such as the seed from the Lodoicea maldivica palm, which are 20 to 24 pounds in weight. One of skill in the art recognizes that seed intended to be used with the embodiments of the invention must be of a size and weight that allow convenient sampling using the methods and apparatus of the present invention. Such seed include, but are not limited to, many agriculturally important seed such as seed from maize (corn), soybean, Brassica species, canola, cereals such as wheat, oats or other grains, and various types of vegetable and ornamental seed. Analogous applications will be obvious from this example and variations obvious to those skilled in the art will be included.

Reference will be made to samples taken from a seed. Sampling methods may be referred to in different terms, such as, for example, sampling, chipping, clipping, slicing, cutting, snipping, or removing a sample. The sample that has been taken can also be referred to using different terms, such as, for example, seed sample, seed tissue sample, seed chip, seed snip, seed sliver, seed clip or clipping, or seed portion.

### B. General Methodology

In one embodiment, this invention utilizes the mechanical clipping of seed in order to create a seed tissue sample for laboratory testing. Seeds are manually held, positioned, and desired portions of the seed are clipped and placed in a tube or other container for testing. The remainder of the seed is also collected and tracked for possible future selection and planting purposes. Seed viability post sampling is an option for the user, and can be affected by where the sample is taken from the seed.

This invention can be utilized to obtain seed samples that are subsequently used for a wide range of analyses, such as, for example, DNA, RNA, protein, spectroscopic and seed composition based assays in which the laboratory results dictate the decisions of which seed to use for breeding, or other experimental purposes.

This methodology addresses the itemized objectives (a)-(f) from the Background of the Invention in at least the following ways.

(a) Viability

Clipping action allows a clip, chip, sliver, or other monolithic piece to be separated from a seed without crushing, tearing, or otherwise destroying the viability of the sampled seed. By using a reasonably sharp cutting edge or edges, and utilizing the mechanical advantage of clipping tools, a non-destructive, usually relatively small part of the seed is removed, leaving the structures and tissues needed to germinate the seed.

(b) Sample Amount

Selection of the clipping tool and method can be commensurate with the size and type of seed so that a minimum sample amount needed for testing can be obtained, but not too much is removed (e.g. an amount which might risk substantial reduction in germination potential). Also, selection of the tool and its manipulation relative the seed allow flexibility and adjustability of amount of sample taken. While this could include manual manipulation of tool and/or seed, it has been found to provide a practical, easily taught, acceptably accurate and rapid sample acquisition method for several applications related to seed sampling.

(c) Sample Location

Similar to sample amount, selection of an appropriately sized and configured clipping tool allows control of location on the seed from which the sample is taken. Again, this can involve some manual action (e.g. holding, positioning, and/or orienting the seed; and operating a hand-held clipping tool), but represents a practical method of individual seed sampling for certain seed sampling applications.

(d) Throughput

Clipping samples from individual seed, one at a time, can appear counter-intuitive to the objective of throughput. However, as discussed above, throughput is not the only consideration in seed sampling for plant breeding. It has been found that even manually operated clipping tools and manual handling of seed can provide a level of throughput that is acceptable for plant breeding applications; especially in light of both the itemized objectives (a)-(f), and also practicalities, including but not limited to cost, complexity, portability, training, support, maintenance, and the like. Whereas automated approaches require large systems that take up considerable space and are very costly, the sampling system of the embodiments is simple, low-cost, and highly adaptable depending on the amount of seed to be sampled and the time available for such sampling. Additional personnel with limited training can quickly be added for sporadic times when high-volume sampling is required. This prevents the need for additional large, costly systems that would sit idle for long periods of time.

(e) Avoiding Contamination

As mentioned, clipping a sample minimizes or eliminates risk of contamination, including cross-contamination between samples. For example, the sample tends to be one piece. Therefore, residual pieces from prior samples are low risk. Furthermore, clipping does not generally produce any dust, debris, or other very small particles that would be difficult to recognize or remove between samplings. This is in contrast to tools such as drilling, milling, grinding, rasping or boring tools, which tend to create such dust or small particles.

Even though there is direct contact between the clipping tool and the seed, risk of contamination between samples is low. Procedures to further reduce such risk can be used. For example, the clipping tool could be cleaned between samples. However, such cleaning is not necessarily required. Some seed is quite hard and no debris or dust is created upon clipping, and no residue is left on the clipping blade(s). In addition, many laboratory assays will be able to discriminate minute amounts of contaminants resulting from previous samples.

(g) Tracking Samples

As indicated previously, mechanical clipping can include manual steps (e.g. seed may be manually held, positioned, and desired portions of the seed clipped by manual actuation of a tool). However, handling of samples (and sampled seed) can include steps that lend to higher throughput and the ability to efficiently and accurately track and correlate samples and/or sampled seed.

For example, a clipped sample can be placed in a tube or other container for storage and/or testing. The remainder of the seed can also be collected and tracked for possible future selection and planting purposes.

A variety of tracking methods known in plant breeding could be used for tracking. A few non-limiting examples include bar codes, RFID tags, printed or handwritten labels, and the like. Storage containers such as indexed seed trays or analogous devices could be used for efficient organization and tracking.

Further illustration of how the itemized objectives and other considerations are factored into seed sampling by clipping will be seen in the specific exemplary embodiments set forth below.

### C. Specific Examples

### 1. Example 1

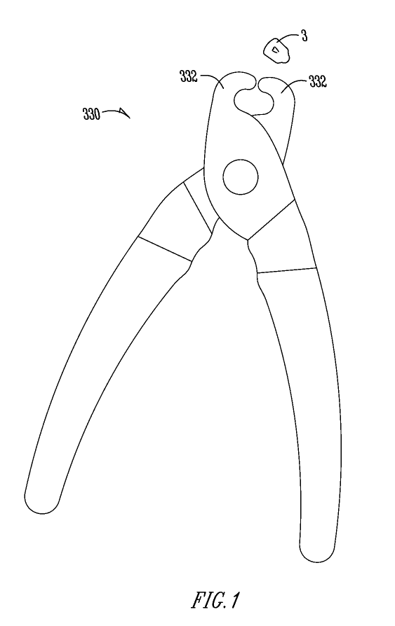

### Individually Clip Sample with Hand-Held and Operated Clipping Tool (FIG. 1)

a) Apparatus

FIG. 1 illustrates a hand-held and operated clipping tool relative a corn seed. The clipping tool 330 is a quite simple, relatively inexpensive, highly portable tool for single seed sampling.

In this example, the clipping tool 330 is a conventional animal nail clipper such as a dog or cat nail clipper. Such clippers are commercially available off-the-shelf from a variety of sources. The opposed jaws 332, each having a sharpened cutting edge, are used to clip off a sample from a corn seed, such as, for example, a sample from the crown of the seed, to obtain a useful quantity of endosperm, e.g., for genetic testing.

An example of such a animal nail clipping tool is a fairly ergonomic, readily available and inexpensive item #743C (heat treated steel with a safety stop bar acting as a optional depth stop for seed size) from Millers Forge, Inc., 1411 Capital Avenue, Plano, Tex. 75974 USA. These types of clippers are available in a variety of styles and sizes (e.g. large dog, cat, etc.). The style generally indicated at FIG. 1 has opposing jaws 332 with concave curved sharpened facing edges, which surround the seed 3 and apply cutting pressure from different sides, including opposed sides, of the seed 3. This tends to avoid applying pressure from only one side, or a predominant amount of pressure from one side. This can deter damage to the seed during cutting. For example, cutting a seed held on a surface with a knife tends to apply pressure from one direction. This has potential to damage, burst, crush or otherwise affect viability of the seed. Also, the facing concave blades present a de facto positioner for the seed relative the blades, at least along one axis. However, single-blade cutting tools can be used.

For corn seed, the clipping tool 330 must be robust enough to handle the reactive forces needed to move the blades through the corn seed, which tends to be a relatively hard seed. Animal nail clippers, such as dog nail clippers, particularly professional grade, should be sufficient. Many of these clippers utilize steel (even surgical steel) and have robust jaws, handles, and pivot.

Several styles of animal nail clippers exist. Most are configured to assist the user to be able to tell with substantial accuracy where the cutting plane will be relative the pet's nail, as it is important not to cut too close to the animal's flesh. The depth stop feature on many animal nail clippers, which is designed to prevent injury to the animal by limiting the depth of nail being cut, is particularly useful in adapting the clippers to seed sampling. The depth stop allows the user to adjust the size of sample being taken from the seed, and allows for consistent, repeatable accuracy in the taking of seed samples from similarly sized seed. Most animal nail clippers are configured to avoid partial clipping, tearing, or pain to the animal. In an analogous sense, sampling of seed needs to have substantial accuracy of amount and location, as well as provide a relatively easy, clean cut. Thus, these types of clippers also resist tearing, crushing, or turning of a seed while cutting to promote a clean, accurate, consistent cut.

FIG. 1 shows a scissor-style clipping tool 330. It works like a pair of scissors. The opposing jaws 332 are two scissor-like notched blades which surround and cut through a nail as the handles are pushed together. These scissor-type nail clippers are not intended for larger, thicker nails, but rather smaller, thinner nails—e.g. lighter jobs, such as very small dogs or cats and bird claws. Scissors are a tool using mechanical advantage to require little force to cut. They are a first-class, double-lever machine with the pivot acting as the fulcrum. Cutting is by applying a local shear stress at the cutting location, which exceeds the material's shear strength.

Pliers-style dog nail clippers are similar to scissor type, but tend to be more robust (e.g. more suited for bigger pet nails). They tend to work in the same fashion as pruning shears. Two notched blades surround and then clip through the nail as handles are squeezed together. Many groomers prefer these dog nail clippers since they make it easy to judge precisely where the blade will be clipping the nail. Pliers-style are essentially heavy-duty dog nail clippers. Some pliers-type cutters cut by indenting and wedging apart the object to be cut, as opposed to shearing action of scissors-type tools. Plier-type cutters may be indicated for bigger and harder seeds.

As can be appreciated, the characteristics of the seed to be sampled can affect which style of clipper to use. Pliers-style, or more robust scissors-style are indicated for corn seed. The size of opening in the blades can vary. An appropriate size should be selected for the type of seed to be sampled. For example, seed much smaller than corn would indicate a smaller scale clipper; perhaps of a size used on smaller animals such as cats, birds, rabbits, and the like.

There are also guillotine-style dog nail clippers, which are also common and widely commercially available, including from sources such as Millers Forge, Inc. (see, e.g., model 744C Pet Nail Trimmer). The dog's nail is inserted into a hole at the top of the trimmer. Handles are squeezed and a blade moves linearly through the nail. It can be cumbersome to get thicker nails into the guide hole. However, such clippers may work acceptably for many seed types and sizes.

However, other types or classes of cutting tools can likewise be used for this seed sampling. Alternatives are discussed later in this document.

b) Operation

(1) Pre-Processing

The only pre-processing of seed 3 is that it be shelled from its ear and singulated.

(2) Orientation

The worker would manually manipulate seed 3 into the desired orientation.

(3) Sample Cutting

The operator of tool 330 would be trained to manually hold the tip cap end of seed 3 in one hand and insert the crown end of seed 3 between the opposing jaws with the jaws in or toward an opened position (as shown in FIG. 1). In this example of tool 330, the opposing jaws 332 have facing cutting edges curved in a concave shape. The open area between them (FIG. 1) de facto serves as a guide or positioner of seed 3 between the jaws. The operator then takes care to position seed 3 relative the cutting plane of the jaws so that only a desirable useful amount of the crown end of seed 3 is aligned with that cutting plane. The concave shaped blades promote a clean cut and good control when cutting the relatively small object, the seed. The nature of the blades also provides a relatively good view of the seed to the operator to promote accuracy of position of cut and amount of sample removed.

The operator manually actuates cutting of seed 3 by convergence of the tool handles in the operator's opposite hand. The operator uses the mechanical advantage of the handles to, in a controlled manner, converge the opposing jaws 332 through seed 3 to cut or slice off the sample.

(4) Collection of Samples

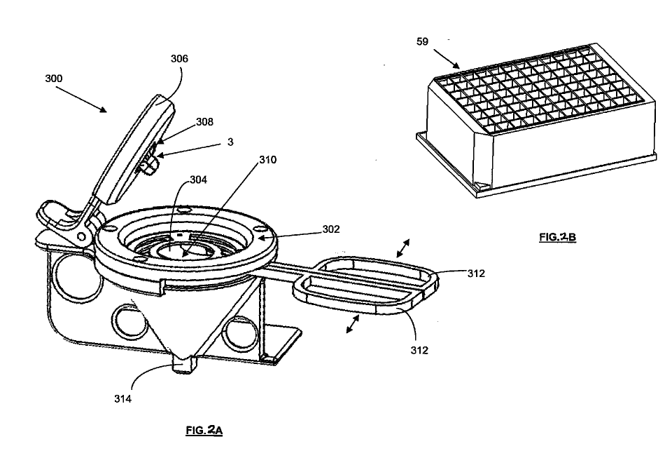

The sample could be collected by hand, on a sheet, or into a bullet tube and transferred to an appropriate well of a sample plate (see example of one type of sample plate or indexing tray 59 in FIG. 2B). Alternatively, it may be workable for the operator to clip the sample off seed 3 and allow it to drop by gravity right into a well of tray 59 of FIG. 2B.

In the present example, the average size of the sample from the crown of a corn seed is between 0.5 and 20 mg. The amount of useful sample can vary according to seed type and application. If corn seed viability after sampling is not required, sample sizes of 30 or 40 mg, or more, would be acceptable and easy to obtain using the embodiments of the invention.

(5) Collection of Cut Seed

Sampled seed could also be manually or otherwise moved to an index tray 59 or similar storage. Sampled seed could be indexed to its sample by placing sample and sampled seed in the same indexed position in each of their trays 59. A label, bar code, or other identifier for each tray could be correlated to track and maintain correlation between samples and seed. As indicated, the identifiers could be machine-readable to allow efficient electronic or computerized storage and retrieval of such information.

This embodiment balances the variety of issues and factors involved with seed sampling in the ways described with respect to the general method above. Controlled clipping of a sample has been shown to not significantly affect the germination potential for the sampled seed. Controlled clipping can provide a useful amount of sample. It can be controlled in size of seed clip or sample removed and location of tissue removal from the seed. It can be controlled to reduce risk of removal of too much sample. Contamination risk is minimized or eliminated by taking a clip of the seed with a clean cut.

(6) Post-Processing

Testing of the samples can occur directly on tray 59 or otherwise, according to methods well known in the art. After test results are obtained, samples and/or seed can be recovered and made available for shipping and use based on the sample test results, e.g., an indication of the presence of a desired trait or characteristic.

Biochemical, genetic, or phenotypic testing of the seed tissue sample can proceed. The cut seed correlated to its sample can then be used accordingly, after determining whether it contains desired biochemical, genetic, or phenotypic traits.

### 2. Example 2

### Individually Clip Sample with Cigar-type Multiple Blade Cutter (FIG. 2A)

a) Apparatus (FIG. 2A)

A hand or table mount single seed sampling tool 300 is shown in FIG. 2A. Funnel-shaped housing or body 302 holds curved cutting blades 310 of a conventional commercially available cigar cutter (e.g. three tangentially moving blades with scissor-type handles) in what will be referred to as a cutting plane. Some cigar cutters have more than three blades.

Such cigar cutters are commercially available. Examples are the XiMTX Multitool available from Xikor of Kansas City, Mo. 64102, and Item 8173B Scissors Cigar Cutter from Cubanoz.biz, Miami Beach, Fla. 33119.

A hinged lid 306 attached to the perimeter of body 302 has a rubber or flexible grommet 308 at its center which is sized to hold a single corn seed 3 when pushed into the center of grommet 308. The geometry of lid 306 and grommet 308 are such that when lid 306 is pivoted down into abutment with body 302, a desired amount of seed 3 (here the crown) extends past the cutting plane of blades 310. The sampling tool 300 is then operated to cut a portion (or sample) of seed 3 off.

b) Operation

(1) Pre-Processing

The only pre-processing of seed 3 is that it be shelled from its ear and singulated so that it can be manually placed in grommet 308.

(2) Singulation

As can be appreciated, the worker will usually keep track of the origin of the singulated seed 3 so that correspondence between each seed, its origin, and the sample can be maintained.

(3) Orientation

The worker would manually manipulate seed 3 into grommet 308 in the desired orientation.

(4) Sample Cutting

When handles 312 are moved apart, the blades move to provide an opening into which seed 3 can be placed. Seed 3 is placed and positioned in that opening when lid 306 is closed. FIG. 2A shows cutter sampling tool 300 with handles 312 moved to the cutting position which closes the gap between the blades 310 in the cutting plane. A seed 3 in the opening in the cutting plane would be cut to separate a portion or sample of seed 3.

As with the embodiment of FIG. 1, this embodiment has cutting edges that surround the seed. When converged, the blades concurrently begin cutting action generally along a single cutting plane from multiple directions for even, precise, clean cutting and deterring damage to the seed.

As discussed previously, it is preferable that the amount of the sample be sufficient for accurate biochemical, genetic, or phenotypic testing and small enough to minimize the effect on germination potential of seed 3 after the sample is separated.

(5) Collection of Samples

In the present example, the average size of the sample was between 0.5 and 20 mg. When cutting the sample, square outlet tube 314 of sampling tool 300 could be placed into (or above) an appropriate well in sample plate 59 to deposit the sample directly into the sample plate 59. The square end of 314 could fit right into the square well to facilitate accurate transfer. The next seed 3 cut could be deposited into the next sample plate well by simply moving sampling tool 300 to the next sample plate well, and so on.

(6) Collection of Cut Seed

The remainder of seed 3 would be retained in grommet 308. It could be directed to individual wells in an appropriate seed plate in the same or correlated well position as its corresponding sample in the sample plate 59.

This embodiment balances the variety of issues and factors involved with seed sampling in some ways similar to prior-described embodiment, and in some ways dissimilar. However, those factors are balanced to achieve at least the following. Controlled cutting of a sample has been shown to not significantly affect the germination potential for the sampled seed. Controlled cutting can provide a useful amount of sample. It can be controlled in size of seed clip or sample removed and location of tissue removal from the seed. It can be controlled to reduce risk of removal of too much sample. A form of seed orientation prior to sampling is disclosed. It uses a receiver to position the seed for controlled cutting with blades. The arrangement also reduces risk of contamination of samples. Some of the structure allows quick and accurate placement of samples in segregated positions in containers to improve throughput of sampling and collection and storage of samples.

(7) Post-Processing

Biochemical, genetic, or phenotypic testing of samples can proceed as previously described. The cut seed correlated to its sample can then be used accordingly after determining whether it contains desired biochemical, genetic, or phenotypic traits.

This embodiment likewise addresses the itemized objectives (a)-(f) as described in the General Method example above.

### D. Options and Alternatives

As can be appreciated by those of ordinary skill in the art, the embodiments of the invention disclosed herein are exemplary only and not comprehensive of the forms they can take. Variations obvious to those skilled in the art will be included within the scope of the invention and its embodiments. Some examples are set forth below.

1. Apparatus

The size, configuration, and materials for the components of the exemplary embodiments can vary according to need and desire.

Optionally, any number of hand or possibly power clipping tools could be incorporated in place of the specific style of clipping tool 330 or sampling tool 300. Examples include, but are not limited to, off-the-shelf or modified versions of cutting devices such as loop scissors or angled scissor style clippers, tin snips, tube cutters, shears, pill cutters or wire cutters. Blades are normally sharp, but usually do not need to be dangerously sharp. Most of these types of tools provide sufficient mechanical advantage and cutting action for sample removal.

Additionally, dual or plural motion cutting edges of the embodiments of FIGS. 1 and 2A-B can be used. But also, single edge cutting blades with static backing plate or rest can work.

As can be appreciated, many tools could be used to cut or snip a sample from the seed. Many reasonably sharp, mechanical advantage tools could be used.

An enhanced system might utilize a sensor (e.g. optical sensor) to sense when the seed is in position relative the cutting blades, and then trigger an actuator that would automatically close the blades and cut the sample.

2. Methods

The precise method steps can vary according to need and desire.

3. Orientation

Some assistance in orientation before clipping of the seed is possible. For example some type of template or receiver could mechanically restrain the seed in a desired orientation which is repeatable between successive seed. Note also that at least some orientation of seed can be manually accomplished by how they are inserted into, for example, a receiver, hole, cup, or other structure. The geometry of such structures can, in some circumstances help orient a seed. This might be particularly true for seed that has a non-symmetrical but consistent shape. Corn is such an example.

4. Post-Processing

Optionally after sample-taking a seed treatment or similar substance could be applied over the area of the seed from which the sample is taken.

For example a substance such as paraffin could be placed or coated over the cut area of a cut seed. There are also commercially available grain sealers that might be used (e.g. Log-Gevity™ product from ABR Products, Inc., Franklin, Wis. USA) to protect against intrusion into a cut seed.

Another example would be to use a seed treatment such as Lockout™ (Becker Underwood Inc.) in order to protect nutrients from eroding from the seed.

Optionally, one or more substances could be applied to the seed after sampling. Examples include, but are not limited to, insecticides, fertilizers or growth enhancers, or anti-fungal agents.

One specific example is bentonite, which is a natural substance that has anti-fungal characteristics. Such substances could be used to increase germination potential and reduce pathogenic attacks on a cut seed. Commercially available chemical seed treatment fungicides include Captan™, a broad-spectrum fungicide, from Drexel Chemical Co., Memphis, Tenn. USA; and Apron™ (metalaxyl) and Maxim™ (fludioxonil) fungicides, from Syngenta, Greensboro, N.C. USA.

5. Analytical Techniques

As can be appreciated by those skilled in the art, the methods by which the samples are processed to derive biochemical, genetic, or phenotypic information can include almost any method known in the art. Many are well-documented and widely known.

6. Applications

a) Type of Seed

The exemplary embodiments and the invention are not limited to corn seed, but can be applied to virtually any seed. Soybeans and canola seed are but two other examples. Other seed of interest include, but are not limited to, seed from canola, sorghum, wheat, sunflower, Brassica species, rice, oats, and other grains, cereals, and other agriculturally significant crops.

b) Uses

Embodiments of the invention can be used in a wide variety of laboratory assays and protocols, and can be applied to various aspects of plant research. Some, but not all, of the ways the invention could be applied include DNA and RNA extraction and testing procedures, genotyping, seed sorting for transgenic seed versus non-transgenic seed, identification of markers, testing for adventitious presence, spectroscopy, food research, oil chemistry and protein biochemistry. This is but a sampling of the methods where the embodiments of the invention find use, and is not intended to be limiting in any way.

What is claimed is:

1. A method of sampling seed comprising:
a. manually positioning the seed in a pre-determined orientation;
b. removing a measurable sample from the seed by manually causing movement of one or more blades through a portion of the seed.

2. The method of claim 1 wherein the removal of the measurable sample from the seed does not cause a significant reduction to germination potential of the seed.

3. The method of claim 1 wherein the seed is corn seed.

4. The method of claim 3 wherein the sample of the seed is taken from the crown end of the seed.

5. The method of claim 1 wherein the step of manually positioning the seed in a pre-determined orientation comprises manually restraining the seed in the pre-determined orientation.

6. The method of claim 1 wherein the movement of one or more blades comprises scissors action or pliers action.

7. An apparatus for sampling an individual seed comprising:
a. a tool comprising
i. a working end including a cutting edge;
ii. a handle operatively connected to the working end to cause movement of the cutting edge;
b. a container for receiving a sample that is separated from the seed by the tool.

8. The apparatus of claim 7 further comprising a means to position and orient the seed relative to the cutting edge.

9. The apparatus of claim 8 wherein the means to position and orient the seed comprises a receiver having a geometry which mechanically restrains the seed in the position and predetermined orientation.

10. The apparatus of claim 7 wherein the tool comprises a tool selected from the group consisting of:
a. a scissors action pet nail clipper;
b. a pliers action pet nail clipper;
c. a tin snip;
d. a tube cutter;
e. a pill cutter;
f. a wire cutter; and
g. a cigar cutter.

11. The apparatus of claim 7 further comprising a container to collect each sampled seed.

12. A method of sampling seed tissue from individual seed comprising:
a. separating a relatively small portion of the seed from the remainder of the seed with a hand-held and operated cutter tool wherein the separating step is adapted to provide a relatively measurable sample of the seed.

13. The method of claim 12 wherein the separating step is adapted to be non-lethal to the remainder of the seed.

14. The method of claim 12 wherein the cutter tool comprises one of:
a. a pet nail clipper;
b. a cigar cutter.

15. The method of claim 12 further comprising collecting the sample to deter contamination of a sample.

16. The method of claim 12 further comprising indexing one or both of the sample and the seed to an indexing system.

17. The method of claim 12 further comprising using the sample in an application comprising biochemical, genetic, or phenotypic testing.

18. The method of claim 17 wherein the biochemical, genetic, or phenotypic testing comprises one or more of:
a. DNA extraction;
b. RNA extraction;
c. genotyping;
d. seed sorting for transgenic versus non-transgenic T1 seed;
e. identification of markers;
f. testing for adventitious presence;
g. spectroscopy;
h. food research;
i. oil chemistry; and
j. protein biochemistry.

19. The method of claim 18 further comprising utilizing the biochemical, genetic or phenotypic testing to make decisions about the use of the remainder seed in a plant advancement experiment.

20. The method of claim 19 wherein the seed comprises corn seed, canola seed, or soybean seed.

21. The method of claim 12 further comprising repeating step a. for successive plural individual seed.

22. A method of sampling plural individual seed comprising:
a. collecting a set of individual seed of known identification or origin;
b. serially sampling individual seed from the set by:
i. clipping a sample of a useful amount from a predetermined location on the seed by manually actuated cutting action;
ii. without creating debris, dust, or particles;
c. tracking each sample in a unique indexed location.

23. The method of claim 22 wherein said method of sampling does not have a significant effect on the germination potential of the sampled seed.

24. The method of claim 22 wherein the clipping comprises forcing one or more blades into the seed generally along a single cutting plane.

25. The method of claim 22 wherein the sample comprises a relatively small percentage of the total volume or weight of the seed.

26. The method of claim 22 further comprising conducting biochemical, genetic, or phenotypic testing on each sample.

27. The method of claim 26 further comprising collecting and tracking sampled seed, correlated to their corresponding samples, for selection or planting purposes.

28. A method of non-lethal sampling for obtaining a useful sample amount from each of a plurality of individual corn seed for physical, chemical, or genetic testing comprising:
a. clipping from each corn seed:
i. a relatively small clip;
ii. from, at or near the crown and away from the embryo;
iii. with a hand-held, manually-operated cutting tool;
iv. without generating significant dust, debris, or contaminating material; and
b. tracking each sample.
